(19) United States
(12) Patent Application Publication
(10) Pub. No.: US 2022/0370157 A1
(43) Pub. Date: Nov. 24, 2022

(54) ROBOTIC KNEE REPLACEMENT PROCEDURE AND INSTRUMENTS

(71) Applicant: Zimmer, Inc., Warsaw, IN (US)

(72) Inventors: Michael Malon, Warsaw, IN (US); Adam H. Sanford, Minneapolis, MN (US); Greg Mangan, Warsaw, IN (US)

(21) Appl. No.: 17/749,102

(22) Filed: May 19, 2022

Related U.S. Application Data
(60) Provisional application No. 63/191,208, filed on May 20, 2021.

(51) Int. Cl. A61B 34/30; A61B 34/10; A61F 2/38

(52) U.S. Cl. CPC A61B 34/30; A61B 34/10; A61F 2/38; A61B 2034/102; A61B 2034/105; A61B 2034/108; A61B 2034/2055

(57) ABSTRACT

A system for performing at least a portion of a robotic knee arthroplasty can include a robotic surgical device including an end effector configured to receive a trial component removably connected thereto. The trial component can be engageable with a resected bone. The system can include a processor communicatively coupled to the surgical robot. The processor can be configured to determine a characteristic of the resected bone. The processor can be configured to plan a placement location of the trial component on the resected bone based on the determined characteristic of the resected bone. The processor can be configured to move the end effector to position the trial component on the resected bone based on the determined characteristic of the resected bone and based on the plan.

### CLAIM OF PRIORITY

This application claims the benefit of U.S. Provisional Patent Application Ser. No. 63/191,208, filed on May 20, 2021, the benefit of priority of which is claimed hereby, and which is incorporated by reference herein in its entirety.

### BACKGROUND

Implants are commonly used to replace various components of a human body, such as bones, bone joints, or tissues. A knee replacement procedure (e.g., knee arthroplasty) can be performed to repair or replace damaged bone or damaged tissue in a patient knee joint. A knee arthroplasty can include repairing or replacing damaged or diseased articular surfaces of the tibia or femur. The arthroplasty procedure can include cutting (e.g., resecting) one or more articular surfaces of the tibia and femur and replacing a portion of each articular surface with a prosthesis (e.g., articular surface implant). A total knee arthroplasty (TKA) can be used to repair all articular surfaces of the tibia and femur, whereas a partial knee arthroplasty (PKA) can be used to repair a portion of the articular surfaces of the knee, such as one of the medial, lateral, or patellofemoral compartment.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 illustrates a perspective view of robotic surgical system including a robotic surgical device and a computer.

FIG. 2 illustrates a perspective view virtual model of a knee including an implant.

FIG. 3A illustrates an isometric view of a trial tool and an instrument adapter.

FIG. 3B illustrates a side view of a trial tool and an instrument adapter.

FIG. 4 illustrates a illustrates a robotic surgical system including a robotic surgical device and instruments.

FIG. 5A illustrates a perspective view of a knee undergoing a knee arthroplasty procedure.

FIG. 5B illustrates a perspective view of a knee undergoing a knee arthroplasty procedure.

FIG. 5C illustrates a perspective view of a knee undergoing a knee arthroplasty procedure.

FIG. 5D illustrates a perspective view of a knee undergoing a knee arthroplasty procedure.

FIG. 6 illustrates a schematic view of a method of operating a robotic surgical system.

FIG. 7A illustrates a top view of a trial tool.

FIG. 7B illustrates a top view of a trial tool and instrument adapter.

FIG. 8 illustrates a block diagram of an example machine upon which any one or more of the techniques discussed herein may be performed.

### DETAILED DESCRIPTION

The TKA and PKA procedures require precise resections of the tibia and femur. The cut depth for each resection is specific to the patient and each prosthesis. A surgeon can validate a resection depth manually by inserting a trial prosthesis and exercising the knee through various motions. However, this resection validation is subjective and subject to errors. Also, when surgeons are placing tibial trials in a knee arthroplasty, surgeons can introduce over and under hang or can otherwise misplace the trial component on the resected tibia. Very small errors in alignment or placement can cause negative or less than ideal outcomes in final implant placement.

This disclosure can help to address these issues by including a quick connect feature on the end effector/instrument interface of a robotic surgical system that can be configured to receive a tibial trial component, or other size-specific profiled instruments for joint replacement surgery, such as sizing plates, sizing plates, or drill guides. The robotic surgical system can place the trial component on the resected tibia at a planned location to a higher accuracy and precision than may be accomplished by a surgeon, which can help to reduce overhang, underhang, or other errors in placement. Further, the robotic surgical system can use the interface between the trial component and the tibial resection to validate the resections made to the tibia to further help to avoid implant placement errors and to help save time by eliminating a validation step following the resection of the tibia.

The above discussion is intended to provide an overview of subject matter of the present patent application. It is not intended to provide an exclusive or exhaustive explanation of the invention. The description below is included to provide further information about the present patent application.

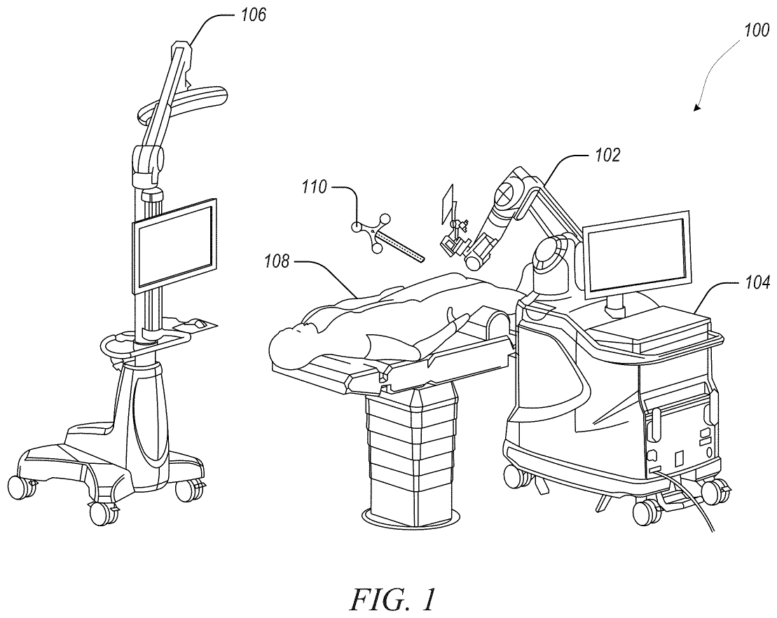

FIG. 1 illustrates a perspective view of a robotic surgical system 100 including a robotic surgical device 102 (e.g., a robot or a robotic arm) and a computing device 104 (e.g., a device having a processor). The robotic surgical device 102 and the computing device 104 can be coupled, such as communicatively coupled or physically connected.

The system 100 can include an optical navigation system 106 that can detect a location of an optical navigation device 110. The optical navigation system 106 can include one or more image capture devices such as a camera, infrared camera, lidar sensor, or the like. The optical navigation system 106 can transmit image streams produced by the image capture devices to the computing device 104 (or control system 104).

The system 100 is shown in relation to a patient 108. The patient 108 can be undergoing a knee arthroplasty procedure (e.g., TKA or PKA). The robotic surgical device 102 can be used to perform aspects of the arthroplasty procedure. A bone or bones of the patient 108 can be modeled before an existing implant is removed. The current bone and implant model can be in a virtual 3D format. For example, frontal and lateral images of the bone and implant can be used to generate a current bone and implant model (e.g., via a front and a lateral x-ray), such as prior to the procedure during a pre-operative planning process or in development of a surgical plan.

In an example, a model of the bone comprises a surface geometry of parts of the bone that are exposed despite the presence of the implant or the limitations of the imaging. The model of the bone can include a surface geometry of the implant relative to adjacent bone surfaces, and a 3D geometry of the implant, for example using a 3D model of the implant (e.g., from the manufacturer, etc.).

The bone modeling can include generating a 3D surface of the bone when the bone modeling is not directly performed by the imaging equipment, or if not complete. In an example in which multiple implants are to be inserted (e.g., an arthroplasty), all bones supporting implants can be modeled. Additional structures can be modeled as well, such as cartilage, hip joint, hip, ankle, or the like.

In terms of planning, an operator can select a position or orientation of a 3D model of an implant that is to be used in an arthroplasty surgery. In another example, the position or orientation can be automatically generated (e.g., using machine learning). Further planning can include determining a location for a cut plane to support the implant(s). The planning can be assisted by an overlay of the revision implants on the bone models.

In an example, tibial tray can be secured to a proximal resected surface of a tibia. The planning can include determining a placement of a tibial implant (e.g., an orientation or position) using the robotic surgical device 102. For example, the robotic surgical device 102 can identify or can use a mechanical axis to determine a plane or location of one or more cuts performed on the tibia. In an example, the resections can be created by the robotic surgical device 102. Additional information including location or orientation of the tibial implants can be used.

The 3D model of the bone with implant(s) can include data pertaining to the surface geometry of a relevant portion of a bone and of the implant, including surfaces of the bone that are exposed despite the presence of the implant. The 3D model of the bone with implant can also include joint line information, full bone models with implants, mechanical axes, center of rotations, etc. The 3D models can also include a bone and implant planning model with an identification of implants that can be used, and bone alteration models to receive the implants and other accessories (e.g., tibial trays and articular components) based on surgical planning.

In an example, the robotic surgical device 102 can be used to cut the bone, for example using a reference guide developed from the 3D model of the bone and the existing implant. The robotic surgical device can autonomously perform the cut (e.g., using the optical navigation system 106 to guide the robotic surgical device 102). The optical navigation system 106 can track the optical navigation device 110, which can be affixed to a bone or an implant of the patient, or affixed to a portion of the robotic surgical device 102. Several optical navigation devices (e.g., trackers) can be used, for example one on each of a femur, tibia, the robotic surgical device 102, and an existing implant. From the tracking information gathered by the optical navigation system 106, used to track each of the optical navigation devices, the robotic surgical device 102 can be guided to perform a cut (e.g., to remove the existing implant). Alternatively, the surgical device 102 can track the bone or an implant of the patient through a registration process of the bone, implant components, etc. Such a registration process can include touching of a sensor to one or more points to determine a location of a component (e.g., bone or implant) in space.

The robotic surgical robot 102 can be used to determine a level of constraint. For example, with a particular amount of laxity detected by the robotic surgical robot 102, a corresponding level of constraint can be used. The level of constraint can be determined based on how much constraint the component system provides due to the loss of ligament or patient anatomy (e.g., hinges are a high level of constraint, posterior stabilized can be a lower level of constraint).

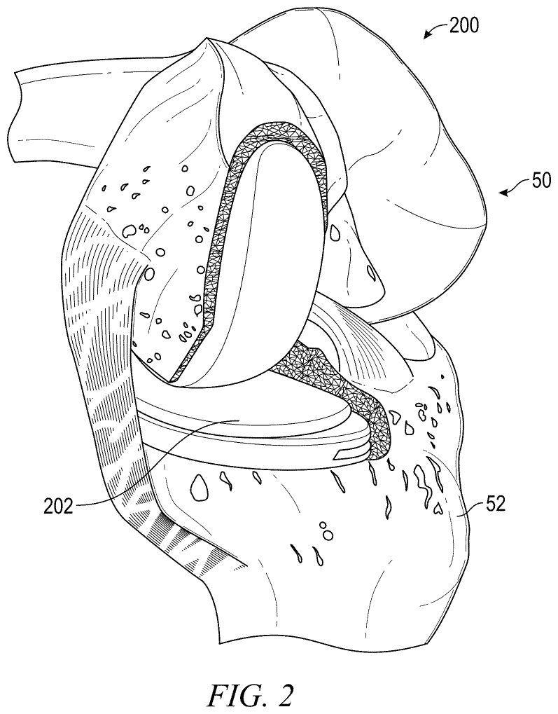

FIG. 2 illustrates an example of a surgical field 200, including an implant 202 affixed to a bone 52 of a knee 50 of a patient undergoing a revision in accordance with at least one example of this disclosure. The surgical field 200 can include a model or an image of the patient. When the surgical field 200 includes a model, the model can be generated using imaging techniques, such as from two x-rays, for example a frontal and a lateral x-ray. These two x-rays can be lined up and a model can be generated using a 3D projection or estimation of the patient anatomy. Other imaging techniques can be used, such as CT scanning (computerized tomography), fluoroscopy, or like radiography methods, for example any that provide suitable resolution of images.

In an example, the patient anatomy can be modeled preoperatively, and used to plan steps of a revision surgical procedure. Deviations from the plan can occur during the procedure, and modifications to the plan (e.g., replanning) can occur intraoperatively, particularly when using a robotic surgical device (e.g., as described above with respect to element 102 of FIG. 1).

In another example, FIG. 2 can include a model generated intraoperatively, for example using registration and optical navigation. This model can be a fully rendered 2D or 3D model of the patient anatomy, or can instead include key points, interpolated or extrapolated points, or other information used for completing a primary or revision joint replacement procedure.

The models described with respect to the patient anatomy need not be actually rendered or displayed. Instead, the models can be used by a robotic surgical device to perform portions of a revision procedure. For example, coordinates of registered points and interpolated or extrapolated other points, simulation of coordinates as moved or cut during a procedure, or the like can be stored in memory. A robotic surgical device can retrieve data stored in the memory when performing a portion of the revision procedure.

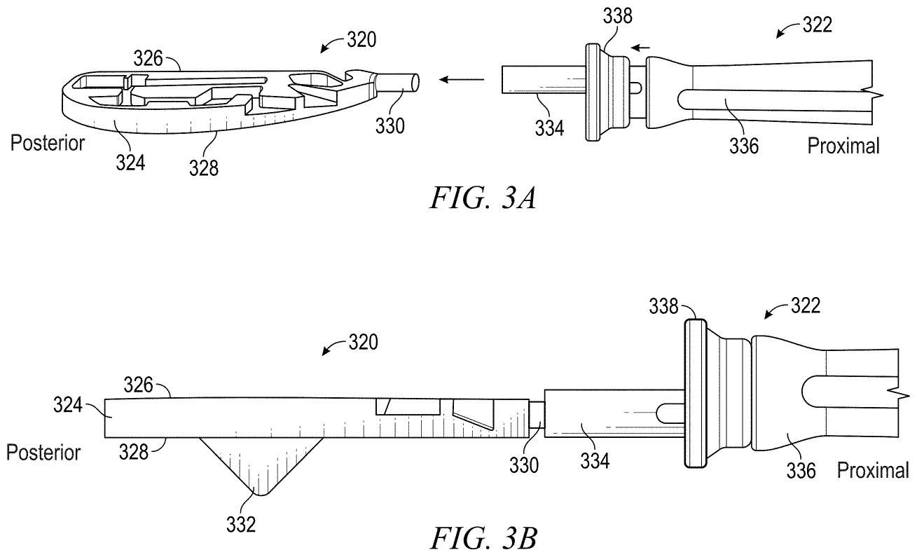

FIG. 3A illustrates a perspective view of a trial tool 320 and an instrument adapter 322. FIG. 3B illustrates a side view of a trial tool 320 and an instrument adapter 322. FIGS. 3A and 3B show orientation indicators Anterior and Posterior. FIGS. 3A and 3B are discussed together below. Applications “QUICK CONNECT FOR ROBOTIC SURGERY,” to Rubrecht, Rodolphe, U.S. Patent Application Ser. No. 63/091,563, filed on Oct. 14, 2020, and “QUICK CONNECT SYSTEM FOR SURGICAL NAVIGATION TOOLS,” to Gaudreau, Jeremie, filed on Mar. 19, 2021, are hereby incorporated by reference herein in entirety.

The trial tool 320 can include a body 324 that can define a top portion 326 and a bottom portion 328. The tool 320 can include a stem 330 connected to the body and extending anteriorly therefrom. The stem 330 can be an elongate member configured to connect to the instrument adapter 322, as shown in FIG. 3B.

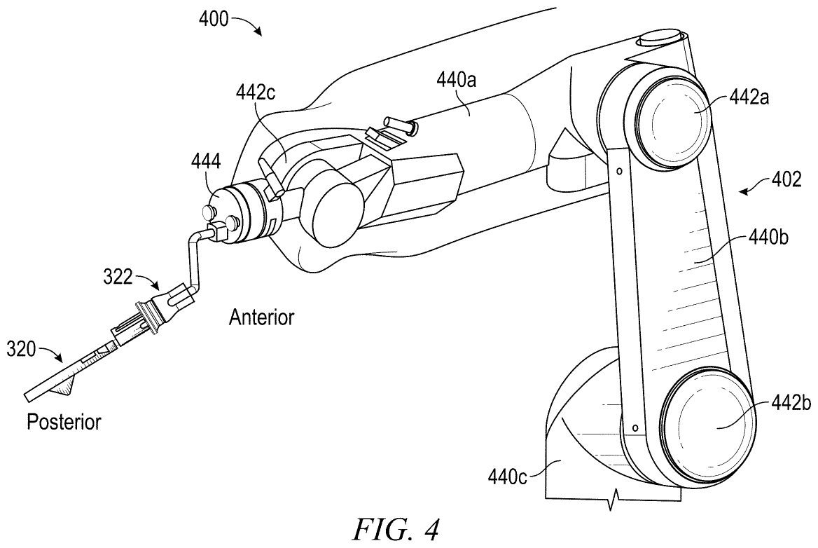

The instrument adapter 322 can include a coupler 334 configured to receive the stem 330 therein. The instrument adapter 322 can be connected to a surgical arm at an anterior end or portion of the adapter 336 (as shown in FIG. 4). The adapter 322 can also include an actuator 338, which can be in the form of a translating collar, but can be other actuators in other examples, such as a button, knob, or the like.

The trial tool 320 and the adapter 322 can be comprised of materials such as metals, plastics, foams, elastomers, ceramics, composites, or combinations thereof. In some examples, the trial tool 320 can be comprised of biocompatible materials such as such as stainless steels, cobalt-chromium, titanium variations, polyether ether ketone (PEEK), polyether ketone ketone (PEKK) or combinations thereof. In one example, the first stem portion 320 can be comprised of PEEK and can be coated with titanium and/or a hydroxyapatite coating.

In operation, the stem 330 of the trial 320 can be inserted into the coupler 334 which can automatically lock the stem 330 to the coupler 334, such as through a biased locking interface (e.g., spring-biased latch or catch). During such an interaction, the actuator 338 can translate anteriorly. Optionally, the actuator 338 can be actuated or operated in other ways, such as by twisting, translating, operating a button, or the like.

When it is desired to disconnect or remove the trial 320 from the adapter 322, the actuator 338 can be operated (such as translated posteriorly, anteriorly, medially, or laterally) to unlock or disengage the coupler 334 from the stem 330. In this way, the trial 320, other trials, or other instruments can be quickly and easily connected and disconnected from a surgical device, such as the surgical device 102.

Optionally, the trial 320 can be adjustable. That is, the body 324 defining the top portion 326 and the bottom portion 328 can be adjustable in one or more dimensions. For example, the body can be adjustable medially and laterally to accommodate tibias of various sizes. Also, optionally, the body 324 can be adjustable anteriorly to posteriorly (or medially to laterally, or laterally to medially, or posteriorly to anteriorly). Such an adjustable trial 320 can help to limit the number of trials that are provided during an arthroplasty procedure.

FIG. 4 illustrates a perspective view of a robotic surgical system 400 including a robotic surgical device 402 and instruments. The robotic surgical device 402 can be similar to the robotic surgical device 102 discussed above. FIG. 4 shows how the robotic surgical device 402 can be connected to the instrument adapter 322 and the trial 320.

The robotic surgical device 402 can include arms or links 440a, 440b, and 440c that can be connected by joints 442a and 442b. The joints 442 can include motors, servos, actuators, joints, or the like. An end effector 444 can be connected to a distal portion of the arm 440a, optionally by a joint 442c. The end effector 444 can be connected to or configured to support the instrument adapter 322 which can be removably or rigidly coupled to the end effector 444.

The joints 442 can be in communication with (e.g., connected to wirelessly or via wire(s)) to a controller (e.g., computing device 104) that can operate the joints 442 (independently or together) to move one or more of the arms 440a, 440b, or 440c to move or position the end effector 444 and therefore the adapter 322 and the trial 320 in space, as desired.

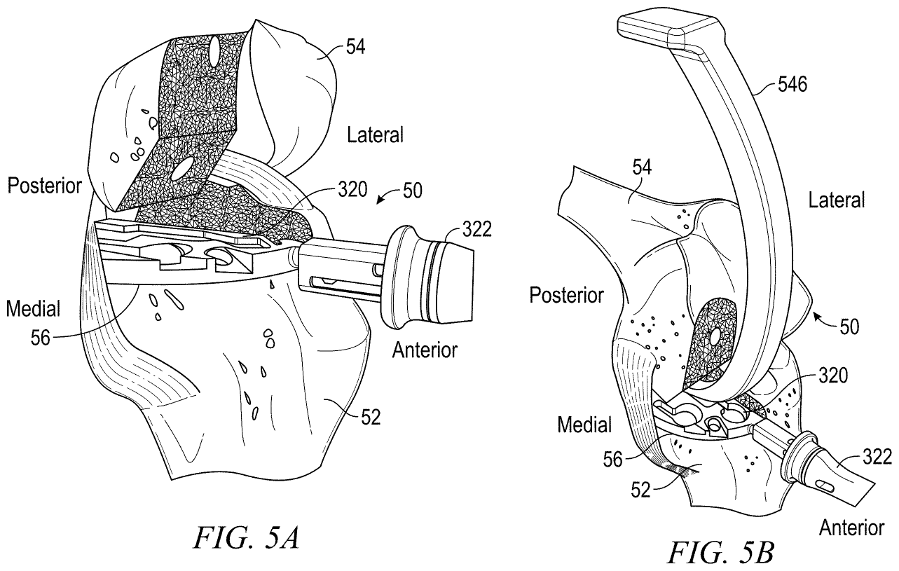

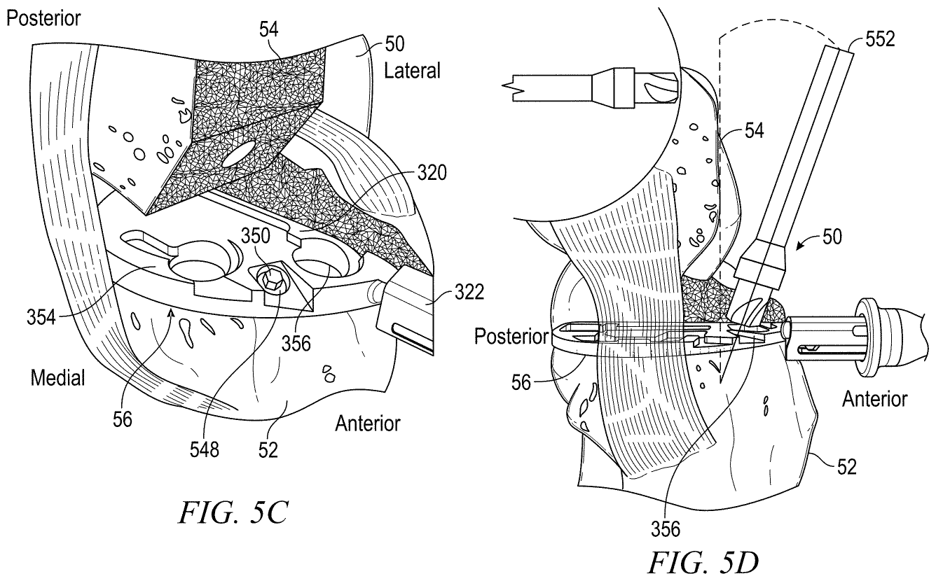

FIG. 5A illustrates a perspective view of a knee 50 undergoing a knee arthroplasty procedure. FIG. 5B illustrates a perspective view of the knee 50 undergoing a knee arthroplasty procedure. FIG. 5C illustrates a perspective view of the knee 50 undergoing a knee arthroplasty procedure. FIG. 5D illustrates a perspective view of the knee 50 undergoing a knee arthroplasty procedure. FIGS. 5A-5D show orientation indicators Anterior and Posterior. FIGS. 5A-5C show orientation indicators Medial and Lateral. FIGS. 5A-5D are discussed together below.

FIG. 5A shows that a robotic surgical system, such as the system 100 or 400, can operate the robotic surgical device, such as the robotic surgical device 102 or 402, to place the tibial trial component 320 on a resected surface 56 of a tibia 52 of a knee 50 of a patient. More specifically, in a procedure, a robotic surgical device (e.g., 102 or 402) can be connected to the instrument adapter 322 such as via an end effector of the surgical device. The instrument adapter 322 can receive the trial component 320, which can be removably coupled or connected to the instrument adapter 322.

At this point, or before this point, a processor of the robotic surgical system can determine a location of the resected surface 56. For example, the computer system 104 can use one or more registration points received thereby, where the reference points can be obtained through contacting a sensor to various locations on the resection 56. The computer system 104 can use data collected from each contact to determine a location of the contact point in space. Multiple contact point determinations can be used by the control system 104 to construct, virtually, or determine a characteristic of the bone or the resected surface 56, such as one or more of a location, shape, or size of the resection 56.

Optionally, the computer system 104 and the optical navigation system 106 can be used to optically track the bone 50 and the effector of the robotic surgical system 102, such as during resection operations, to determine a shape, size, and location of the resection 56. A model, such as the model 200, can be updated based on the position and movements of the surgical system 102 during the resection to determine the size and shape of the resection 56 and to update the model to reflect the actual resection 56.

Whether the resected surface 56 characteristic is determined using optical tracking or registrations points, the determined location 56 can be used to update or modify the model to reflect the actual resection 56. Once the model is updated, the computer system 104 can use the model or the measured or determined location and shape of the resection to plan a placement location of the trial component 320 on the resected surface 56 on the bone 52.

The control system 104 can then operate the surgical device 102 (e.g., the surgical arm 402) to move the end effector 440 to position the trial component 320 on the resected surface 56 on the bone 52 based on the determined location of the resected bone and based on the plan. By placing the trial component 320 in a location using the control system 104 based on the plan or the determined location, the system 100 can help to limit overhang, under-hang, or other placement errors, while also maintaining familiar conventional instrumentation (i.e., the trial component 330).

Because the robotic surgical device 102 is utilized to place the trial component 320 and because an entire surface of the trial component 320 can be known by the system (such as through a database including dimensions and/or virtual 3D models of one or more trial components). A comparison between the inferior surface of the trial component 320 (e.g., the surface intended to engage the resected surface 56) and the determined location of the resected bone (e.g., the virtual model of the resected surface generated by a point cloud or other means) can be used by the control system 104 to determine an understanding of how the trial component 320 should interact with the resected surface 56 of the tibia 52.

Then, an interaction between the trial component 320 and the resected surface 56 can be observed by the control system (such as via the optical navigation system 106) such as where the trial component 320 (e.g., interior surface) contacts the resected surface 56. The interaction between the trial component 320 and the resected surface 56 can be compared to the expected interaction and the control system 104 can determine whether the resection was made properly. Such a determination can be used to guide an update to the resection or can be used to validate the resection before proceeding to further use of the trial component, such as use of a drill guide.

Optionally, the trial component 320 can be a sensor or can include one or more sensors embedded therein, as shown in “KNEE ARTHROPLASTY VALIDATION AND GAP BALANCING INSTRUMENTATION,” to Gogarty, Emily, U.S. Patent Application Ser. No. 63/126,395, filed on Dec. 16, 2020, or as shown in “SENSING FORCE DURING PARTIAL AND TOTAL KNEE REPLACEMENT SURGERY,” to Fisher, Michael, filed on Aug. 20, 2008, which are hereby incorporated by reference herein in entirety. The sensors of the trial component 320 can be in communication with the control system 104 and can transmit data collected by the sensor(s) to the control system 104, where the control system 104 can be used to determine a location, size, or shape of the bone, for example.

Optionally, when the trial component 320 includes one or more sensors, the trial component can be inserted into a position to engage the resected surface 56 without having an updated model (or virtual model) of the bone or without planning an exact final location of the trial 320 with respect to the resected surface 56. The trial component 320 can then be moved to engage the resected surface 56 and can produce a sensor signal based on engagement between the trial component 320 (and its sensors) with the bone (such as the resected surface 56). Such a signal can be transmitted from the trial 320 to the control system 104, where the control system 104 can use the signal (and optionally other information, such as a model) to determine a location, shape, or size of the bone or resected surface 56.

Though FIG. 5A shows the trial component 320 as being for a partial knee replacement, a trial component for a total knee replacement can also be placed by the robot surgical device 102 or 402.

FIG. 5B shows that an impactor 540 can be engaged with the trial component 320. The impactor 546 can be impacted to drive the keel 332 into the tibia 52 to secure the trial 320 to the resected surface 56. The instrument adapter 322 can remain connected to the trial 320 during impaction, where the control system 104 can operate the robotic surgical system 102 to maintain the position of the trial 320 on the resected surface 56 during impaction, such as by using the model and the determined location and shape of the resected surface 56 to help prevent the trial 320 from moving with respect to the resected surface 56 during impaction.

Optionally, as shown in FIG. 5C, a fastener 548 can be driven through a bore 350 of the trial to further secure the trial 330 to the bone 52. The instrument adapter 322 can remain connected to the trial 320 during driving operations, where the control system 104 can operate the robotic surgical system 102 to maintain the position of the trial 320 on the resected surface 56 during driving operations, such as by using the model and the determined location and shape of the resected surface 56 to help prevent the trial 320 from moving with respect to the resected surface 56 during driving operations.

As shown in FIG. 5D, a drill 552 can be used to drill through bores 354 and 356 of the trial 330 to create bores or holes for protrusions or pegs of the tibial implant. The instrument adapter 322 can remain connected to the trial 320 during driving operations, where the control system 104 can operate the robotic surgical system 102 to maintain the position of the trial 320 on the resected surface 56 during drilling operations, such as by using the model and the determined location and shape of the resected surface 56 to help prevent the trial 320 from moving with respect to the resected surface 56 during drilling operations.

Following completion of the tibia preparations (or those using the trial 320), the trial 320 can be removed from the resected surface 56, such as where the control system 104 can operate the robotic surgical device 102 to move the instrument adapter 322 to remove the trial 320 from the resected surface 56 so that the remainder of the arthroplasty procedure can be completed.

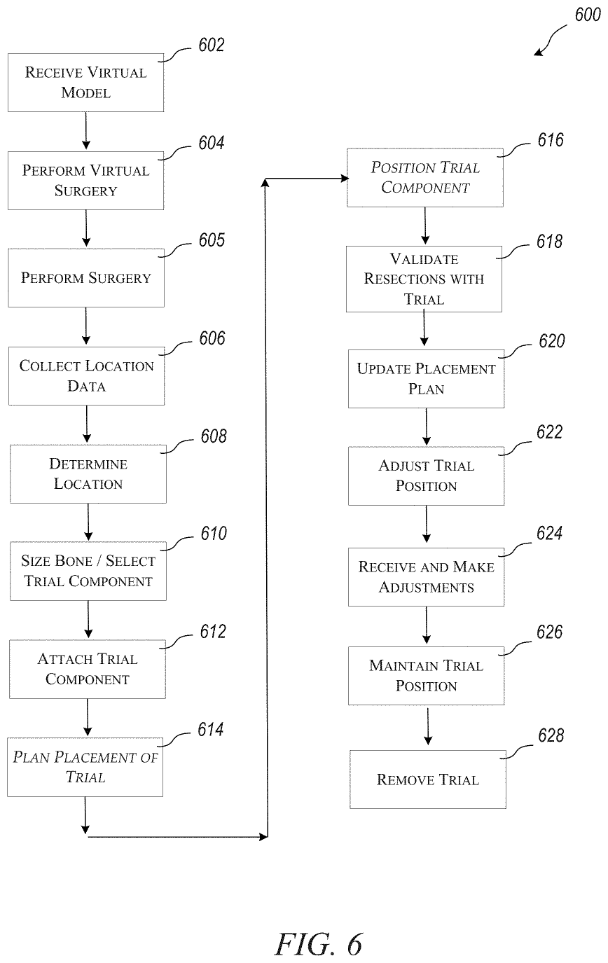

FIG. 6 illustrates a schematic view of a method 600 of operating a robotic surgical system. The method 600 can be a method of operating a robot surgical system to place a trial component on a resected tibial portion. More specific examples of the method 600 are discussed below. The steps or operations of the method 600 are illustrated in a particular order for convenience and clarity; many of the discussed operations can be performed in a different sequence or in parallel without materially impacting other operations. The method 600 as discussed includes operations performed by multiple different actors, devices, and/or systems. It is understood that subsets of the operations discussed in the method 600 can be attributable to a single actor, device, or system could be considered a separate standalone process or method.

At step 602, a virtual model of the bone can be received or produced. For example the model 200 can be received or produced by the control system 104. At step 604 a virtual surgery can be performed on virtual model of the bone based on a pre-surgical plan. For example, the model 200 can be resected and implants can be installed, virtually, on the model 200 by the control system. Optionally, the virtual surgery can be performed by another system and can be delivered to the control system 104.

At step 605, various steps of the surgical procedure can be performed. For example, an opening can be created adjacent the patient's knee joint and soft tissues can be moved, resected, or positioned for access to a distal portion of the patient's femur and a proximal portion of the patient's tibia. Also, the surgical system 102 can be used to perform one or more cuts or resections of the femur or tibia. For example, the tibia 52 can be resected to create the resected surface 56 on a proximal portion of the tibia.

At step 606 location data can be collected for use in selecting and virtually positioning implants. Operation 606 is discussed in term of optional steps 606a and 606b, which can be performed as alternatives or together to collect location data. At step 606a location data can be collected via registration points of the resected bone can be received intraoperatively. For example, a sensor connected to the robotic surgical system 102 can produce reference points transmitted to the control system 104. Optionally or additionally, at step 606b, location data can be collected during the resection of the bone can be received intraoperatively via an image stream including imagery of the surgical system, where the optical navigational system 106 can track optical navigational markers 110 connected to the bone and to the surgical arm or its instruments. For example, the optical navigation system 106 can produce an image stream that can be transmitted to the control system 104 where the control stream includes imagery of a navigational marker 110 connected to the tibia 52 of the patient and a navigational marker connected to the arm links 440 or the end effector 444. At step 608, a location of a resected bone can be determined. For example, the location of the resected bone can be determined based on the registration points or based on the optical data.

At step 610, the bone (such as a proximal portion of a tibia) can be sized. The bone can be sized using a registration process or by updating the virtual model based on tracking of the bone and the tool interfacing therewith (e.g., visual tracker and cutting device). Once the bone is sized, a trial component can be selected from a plurality of trial components of various sizes based on the location or size of the resected bone. For example, the trial component 320 can be selected based on the model 200, the determined location of the resection 56, or the determined size of the bone 52 at the resection 56. At step 612, a trial can be attached to an end effector of a robotic surgical device. For example, the trial component 320 can be attached to the instrument adapter 322 of the surgical device 102 or 402. Optionally, where the trial component is adjustable, the trial need not be selected at step 610 and can be adjusted instead to match a size of the patient's proximal resected tibia. For example, the trial 320 can be adjusted medially and laterally and can be adjusted posteriorly and anteriorly to match the size and shape of the proximal resected tibia.

At step 614, a placement location of the trial component 320 on the resected surface 56 of the bone 52 can be planned by the control system 104 based on the determined location of the resected bone, or the determined size of the bone, or the model. At step 616, the surgical device 102 or 104 can be operated to move the end effector 444 to position the trial component 320 on the resected surface 56 of the bone 52 based on the determined location of the resected surface 56 of the bone 52, or the determined size of the resected surface 56 of the bone 52, or the trial placement plan.

At step 618 a shape and size of the resected bone can be determined based on engagement between the trial component and the resected bone and based on the determined location of the resected bone such as to validate the previously made bone resections. For example, a shape or size of the resected surface 56 of the bone 52 can be determined based on engagement between the trial component 320 and the resected surface 56. That is, the control system 104 can detect or determine the location of the trial component 320 and can receive feedback, such as force sensor feedback, to determine a location of the trial 320 when it engages the resected surface 56. The control system 104 can compare the location of the trial component's engagement with the resected surface 56 to the plan (such as using the model on which virtual surgery was performed). Using the comparison, the control system 104 can validate the resections made to form the resected surface 56.

Optionally, when the resected surface 56 shape, size, or location of the resection 56 is determined by the control system 104, the control system 104 can produce a notification that the resection is not within a specified tolerance (e.g., angle, position, or size) of the planned or virtual resection. The system 104 can then update the surgical plan and instruct a user, such as through producing a display, that the resection must be modified to the new surgical plan. At this point, the surgeon or user can perform steps to modify the resected surface 56. Following the updated resection procedure, steps 605 through 618 can be repeated to validate the size, shape, or location of the resected surface 56.

At step 620, the plan can be updated where the control system 104 determines a variation in the resection from the plan. In an example where the resected surface 56 varies from expectations, the placement plan can be updated based on the plan and based on the determined shape of the resected surface 56 of the bone 52 and the location of the resected surface 56 of the bone 52. Optionally, when the placement plan is updated by the control system 104, a new trial can be selected that better matches the shape and size of the resected surface 56. Then, at step 622 the surgical system 102 can be operated to move the end effector 444 to position the trial component 320 on the resected surface 56 of the bone 52 based on the updated placement plan.

At step 624, the control system 104 can receive instructions to modify overhang of the trial component on the resected bone in an overhang location. For example, a physician can check placement of the trial component 320 and can compare placement to the virtual model. The physician can provide input to the control system 104 to adjust the placement in one or more overhang locations. The placement plan can be updated based on the overhang locations received and the surgical system 102 can be operated by the control system 104 to move the trial component 320 based on the updated placement plan.

At step 626, following placement of the trial component, the trial component can be impacted, such as to temporarily secure the trial component to the tibia, such as via a keel or bone spike. Also, the trial component may be used as a guide for a drilling operation to drill one or more bores in the tibia. Similarly, the trial component can be used as a guide for pin placement. Optionally, at step 626, the surgical arm can be operated to maintain a position of the trial component with respect to the resected bone during a drilling, pinning, or impacting operation performed on or through the trial component. Maintaining the trial component in its position relative to the tibia or the resected surface 56 can help to increase bores created in a drilling operation, can help to increase accuracy of placement location of a pin, or can help to increase placement (securing) accuracy of the trial component to the tibia.

At step 628, the control system 104 can operate the surgical system 102 to move the end effector 444 to remove the trial component 320 from the resected surface 56 of the bone 52 based on the determined location of the resected bone and the plan.

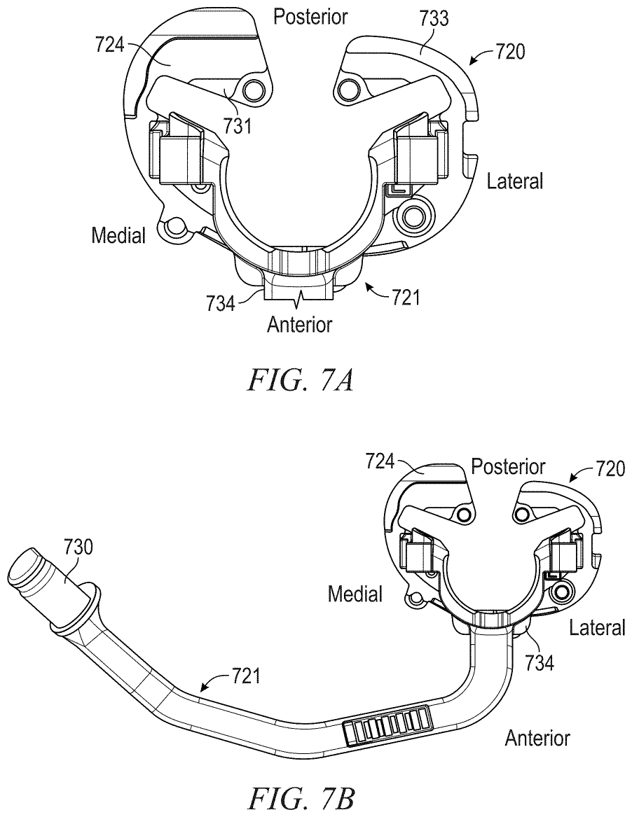

FIG. 7A illustrates a top view of a trial tool 720. FIG. 7B illustrates a top view of a trial tool and arm 721. FIGS. 7A and 7B show orientation indicators Medial, Lateral, Anterior, and Posterior. FIGS. 3A and 3B are discussed together below.

The trial tool 720 can include a body 724 that can define a top portion and a bottom portion. The body 724 can include a medial portion 731 engageable with a medial portion of a tibia and the body 724 can include a lateral portion 733 engageable with a lateral portion of the tibia. The trial tool 720 and the arm 721 (and connected components) can be comprised of materials such as metals, plastics, foams, elastomers, ceramics, composites, or combinations thereof.

A stem 730 can be connected to the arm 721 and can extend therefrom. The stem 730 can be an elongate member configured to connect to the instrument adapter 322, shown in FIG. 3B. A coupler 734 can be connected to the arm 721 and configured engage the body 724 to releasably secure the arm 721 to the trial 720. Optionally, the coupler 734 can be disengaged from the trial 720 to disengage the arm 721 from the trial 720 such as after securing the trial 720 to a bone. Alternatively, the arm 721 can be connected to trial components of various sizes, allowing the arm 721 to be used with whichever trial is determined to fit the bone, helping to reduce instrument cost where multiple trial components are used in a kit.

In operation, the trial tool 720 can be used similarly to the trial 320, but for a total knee arthroplasty. For example, the trial tool 720 can be used in steps for preparing the keel for insertion into the tibia, such as for a broaching operation. During such an operation, the arm 721 can remain connected to the trial 720 during broaching, where the control system 104 can operate the robotic surgical system 102 to maintain the position of the trial 720 on the resected surface 56 during broaching, such as by using the model and the determined location and shape of the resected surface 56 to help prevent the trial 720 from moving with respect to the resected surface 56 during impaction. Maintaining the trial 720 in its position relative to the tibia or the resected surface 56 can help to increase accuracy of the broaching operation.

A similar procedure can be followed for other operations using the trial 720 on a total knee arthroplasty, partial knee arthroplasty, or revision arthroplasty procedure. For example, the trial 720 can be used in steps for cutting operations or chiseling operations of a tibia, such as in preparation for receiving the keel of the implant. Maintaining the trial 720 in its position relative to the tibia or the resected surface 56 for cutting or chiseling operations can help to increase accuracy of the cutting or chiseling operations.

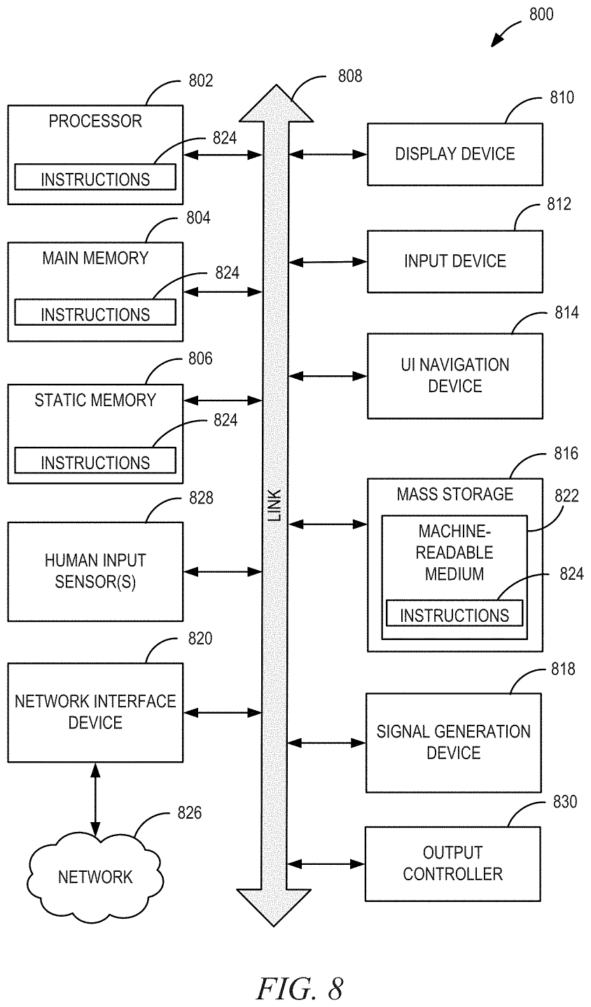

FIG. 8 illustrates a block diagram of an example machine 800 upon which any one or more of the techniques discussed herein can perform in accordance with some embodiments. In alternative embodiments, the machine 800 can operate as a standalone device or can be connected (e.g., networked) to other machines. In a networked deployment, the machine 800 can operate in the capacity of a server machine, a client machine, or both in server-client network environments. In an example, the machine 800 can act as a peer machine in peer-to-peer (P2P) (or other distributed) network environment. The machine 800 can be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 800 can include a hardware processor 802 (e.g., a central processing unit (CPU), a graphics processing unit (GPU), a hardware processor core, or any combination thereof), a main memory 804 and a static memory 806, some or all of which can communicate with each other via an interlink (e.g., bus) 808. The machine 800 can further include a display unit 810, an alphanumeric input device 812 (e.g., a keyboard), and a user interface (UI) navigation device 814 (e.g., a mouse). In an example, the display unit 810, input device 812 and UI navigation device 814 can be a touch screen display. The machine 800 can additionally include a storage device (e.g., drive unit) 816, a signal generation device 818 (e.g., a speaker), a network interface device 820, and one or more sensors 821, such as a global positioning system (GPS) sensor, compass, accelerometer, or other sensor. The machine 800 can include an output controller 828, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

The storage device 816 can include a machine readable medium 822 on which is stored one or more sets of data structures or instructions 824 (e.g., software) embodying or utilized by any one or more of the techniques or functions described herein. The instructions 824 can also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 can constitute machine readable media.

While the machine readable medium 822 is illustrated as a single medium, the term “machine readable medium” can include a single medium or multiple media (e.g., a centralized or distributed database, and/or associated caches and servers) configured to store the one or more instructions 824. The term “machine readable medium” can include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine readable medium examples can include solid-state memories, and optical and magnetic media.

The instructions 824 can further be transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 820 can include one or more physical jacks (e.g., Ethernet, coaxial, or phone jacks) or one or more antennas to connect to the communications network 826. In an example, the network interface device 820 can include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software.

### Notes and Examples

The following, non-limiting examples, detail certain aspects of the present subject matter to solve the challenges and provide the benefits discussed herein, among others.

Example 1 is a system for performing at least a portion of a robotic knee arthroplasty, the system comprising: a robotic surgical device including an end effector configured to receive a trial component removably connected thereto, the trial component engageable with a resected bone; and a processor, communicatively coupled to the surgical robot, the processor configured to: determine a characteristic of the resected bone; plan a placement location of the trial component on the resected bone based on the determined characteristic of the resected bone; and command the surgical device to move the end effector to position the trial component on the resected bone based on the planed placement location.

In Example 2, the subject matter of Example 1 optionally includes wherein the processor is further configured to: determine a shape of the resected bone based on engagement between the trial component and the resected bone and based on the determined characteristic of the resected bone.

In Example 3, the subject matter of any one or more of Examples 1-2 optionally include wherein the processor is further configured to: receive a virtual model of the bone; and perform a virtual resection on virtual model of the bone based on a pre-surgical plan.

In Example 4, the subject matter of Example 3 optionally includes wherein the processor is further configured to: determine a shape of the resected bone based on engagement between the trial component and the resected bone, based on the determined characteristic of the resected bone, and based on the virtual surgery.

In Example 5, the subject matter of Example 4 optionally includes wherein the processor is further configured to: update the placement plan based on the determined shape of the resected bone and the virtual surgery.

In Example 6, the subject matter of Example 5 optionally includes wherein the processor is further configured to: operate the surgical arm to move the end effector to position the trial component on the resected bone based on the updated placement plan.

In Example 7, the subject matter of any one or more of Examples 1-6 optionally include wherein the processor is further configured to: operate the surgical arm to maintain a position of the trial component with respect to the resected bone during a drilling, pinning, or impacting operation performed on or through the trial component.

In Example 8, the subject matter of any one or more of Examples 1-7 optionally include wherein the processor is further configured to: select the trial component from a plurality of trial components of various sizes based on the characteristic of the resected bone.

In Example 9, the subject matter of any one or more of Examples 1-8 optionally include wherein the processor is further configured to: determine an overhang of the trial component on the resected bone based on the characteristic of the resected bone.

In Example 10, the subject matter of any one or more of Examples 1-9 optionally include wherein the processor is further configured to: receive instructions to modify overhang of the trial component on the resected bone in an overhang location; and update the placement plan based on the overhang location.

In Example 11, the subject matter of any one or more of Examples 1-10 optionally include wherein the processor is further configured to: intraoperatively receive reference points of the resected bone; and determine the characteristic of the resected bone based on the reference points.

In Example 12, the subject matter of any one or more of Examples 1-11 optionally include wherein the processor is further configured to: intraoperatively receive an image stream including imagery of optical navigation devices of the surgical system during the resection of the bone; and determine the characteristic of the resected bone based on the image stream.

In Example 13, the subject matter of any one or more of Examples 1-12 optionally include wherein the processor is further configured to: command the surgical arm to move the end effector to remove the trial component from the resected bone based on the determined location of the resected bone and the plan.

In Example 14, the subject matter of any one or more of Examples 1-13 optionally include wherein the resected bone is a proximal portion of a tibia.

Example 15 is a method for performing at least a portion of a robotic knee arthroplasty, the method comprising: attaching a trial component to an end effector of a robotic surgical device; determining a characteristic of a resected bone; plan a placement location of the trial component on the resected bone based on the determined characteristic of the resected bone; and operating the surgical device to move the end effector to position the trial component on the resected bone based on the determined characteristic of the resected bone and the plan.

In Example 16, the subject matter of Example 15 optionally includes determining a shape of the resected bone based on engagement between the trial component and the resected bone and based on the determined characteristic of the resected bone.

In Example 17, the subject matter of any one or more of Examples 15-16 optionally include receiving a virtual model of the bone; and performing a step of a virtual surgery on virtual model of the bone based on a pre-surgical plan.

In Example 18, the subject matter of Example 17 optionally includes determining a shape of the resected bone based on engagement between the trial component and the resected bone, based on the determined characteristic of the resected bone, and based on the virtual surgery.

In Example 19, the subject matter of Example 18 optionally includes updating the placement plan based on the determined shape of the resected bone and the virtual surgery.

In Example 20, the subject matter of Example 19 optionally includes command the surgical arm to move the end effector to position the trial component on the resected bone based on the updated placement plan.

In Example 21, the subject matter of any one or more of Examples 19-20 optionally include operate the surgical arm to maintain a position of the trial component with respect to the resected bone during a drilling, pinning, or impacting operation performed on or through the trial component.

In Example 22, the subject matter of any one or more of Examples 15-21 optionally include select the trial component from a plurality of trial components of various sizes based on the characteristic of the resected bone.

In Example 23, the subject matter of any one or more of Examples 15-22 optionally include receive instructions to modify overhang of the trial component on the resected bone in an overhang location; update the placement plan based on the overhang location.

In Example 24, the subject matter of any one or more of Examples 15-23 optionally include intraoperatively receive reference points of the resected bone; and determine the characteristic of the resected bone based on the reference points.

In Example 25, the subject matter of any one or more of Examples 15-24 optionally include intraoperatively receive an image stream including imagery of the resected bone; and determine the characteristic of the resected bone based on the image stream.

In Example 26, the subject matter of any one or more of Examples 15-25 optionally include operating the surgical arm to move the end effector to remove the trial component from the resected bone based on the determined characteristic of the resected bone and the plan.

In Example 27, the subject matter of any one or more of Examples 15-26 optionally include wherein the resected bone is a proximal portion of a tibia.

Example 28 is a non-transitory machine-readable medium including instructions, for pre-operatively developing a reverse shoulder arthroplasty plan, which when executed by a machine, cause the machine to: attach a trial component to an end effector of a robotic surgical device; determine a location of a resected bone; plan a placement location of the trial component on the resected bone based on the determined location of the resected bone; and operate the surgical device to move the end effector to position the trial component on the resected bone based on the determined location of the resected bone and the plan.

In Example 29, the apparatuses or method of any one or any combination of Examples 1-28 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A system for performing at least a portion of a robotic knee arthroplasty, the system comprising:
a robotic surgical device including an end effector configured to receive a trial component removably connected thereto, the trial component engageable with a resected bone; and
a processor, communicatively coupled to the surgical robot, the processor configured to:
determine a characteristic of the resected bone;
plan a placement location of the trial component on the resected bone based on the determined characteristic of the resected bone; and
command the surgical device to move the end effector to position the trial component on the resected bone based on the planed placement location.

2. The system of claim 1, wherein the processor is further configured to:
determine a shape of the resected bone based on engagement between the trial component and the resected bone and based on the determined characteristic of the resected bone.

3. The system of claim 1, wherein the processor is further configured to:
receive a virtual model of the bone; and
perform a virtual resection on virtual model of the bone based on a pre-surgical plan.

4. The system of claim 3, wherein the processor is further configured to:
determine a shape of the resected bone based on engagement between the trial component and the resected bone, based on the determined characteristic of the resected bone, and based on the virtual surgery.

5. The system of claim 4, wherein the processor is further configured to:
update the placement plan based on the determined shape of the resected bone and the virtual surgery.

6. The system of claim 5, wherein the processor is further configured to:
operate the surgical arm to move the end effector to position the trial component on the resected bone based on the updated placement plan.

7. The system of claim 1, wherein the processor is further configured to:
operate the surgical arm to maintain a position of the trial component with respect to the resected bone during a drilling, pinning, or impacting operation performed on or through the trial component.

8. The system of claim 1, wherein the processor is further configured to:
select the trial component from a plurality of trial components of various sizes based on the characteristic of the resected bone.

9. The system of claim 1, wherein the processor is further configured to:
determine an overhang of the trial component on the resected bone based on the characteristic of the resected bone.

10. The system of claim 1, wherein the processor is further configured to:
receive instructions to modify overhang of the trial component on the resected bone in an overhang location; and
update the placement plan based on the overhang location.

11. The system of claim 1, wherein the processor is further configured to:
intraoperatively receive reference points of the resected bone; and
determine the characteristic of the resected bone based on the reference points.

12. The system of claim 1, wherein the processor is further configured to:
intraoperatively receive an image stream including imagery of optical navigation devices of the surgical system during the resection of the bone; and
determine the characteristic of the resected bone based on the image stream.

13. The system of claim 1, wherein the processor is further configured to:
command the surgical arm to move the end effector to remove the trial component from the resected bone based on the determined location of the resected bone and the plan.

14. The system of claim 1, wherein the resected bone is a proximal portion of a tibia.

15. A method for performing at least a portion of a robotic knee arthroplasty, the method comprising:
attaching a trial component to an end effector of a robotic surgical device;
determining a characteristic of a resected bone;
plan a placement location of the trial component on the resected bone based on the determined characteristic of the resected bone; and
operating the surgical device to move the end effector to position the trial component on the resected bone based on the determined characteristic of the resected bone and the plan.

16. The method of claim 15, further comprising:
determining a shape of the resected bone based on engagement between the trial component and the resected bone and based on the determined characteristic of the resected bone.

17. The method of claim 15, further comprising:
receiving a virtual model of the bone; and
performing a step of a virtual surgery on virtual model of the bone based on a pre-surgical plan.

18. The method of claim 17, further comprising:
determining a shape of the resected bone based on engagement between the trial component and the resected bone, based on the determined characteristic of the resected bone, and based on the virtual surgery.

19. The method of claim 18, further comprising:
updating the placement plan based on the determined shape of the resected bone and the virtual surgery.

20. The method of claim 19, further comprising:
command the surgical arm to move the end effector to position the trial component on the resected bone based on the updated placement plan.
